(19) United States
(12) Patent Application Publication
(10) Pub. No.: US 2016/0151254 A1
(43) Pub. Date: Jun. 2, 2016

(54) USE OF SILICON-CONTAINING PARTICLES FOR PROTECTION FROM UV RADIATION, PROCESS FOR PRODUCTION THEREOF AND FORMULATIONS COMPRISING THEM

(71) Applicants: Juergen Erwin LANG, Karlsruhe (DE); Michael KROELL, Linsengericht (DE); Goswin UEHLENBRUCK, Oberursel (DE)

(72) Inventors: Juergen Erwin LANG, Karlsruhe (DE); Michael KROELL, Linsengericht (DE); Goswin UEHLENBRUCK, Oberursel (DE)

(73) Assignee: Evonik Degussa GmbH, Essen (DE)

(21) Appl. No.: 14/951,034

(22) Filed: Nov. 24, 2015

(30) Foreign Application Priority Data
Nov. 28, 2014 (EP) 14195302

(51) Int. Cl. A61K 8/25; A61K 8/23; A61K 8/02; A61Q 17/04

(52) U.S. Cl. CPC A61K 8/25; A61Q 17/04; A61K 8/23; A61K 8/0245; A61K 2800/805; A61K 2800/413; A61K 2800/262; A61K 2800/26

(57) ABSTRACT

Silicon-containing particles are used for protection of human cells from electromagnetic radiation in the UV range and optionally in the visible as far as the IR range, where the particles preferably take the form of clusters of primary particles having a particle size in the range from 5 to 100 nm. A particular advantage is the possibility of matching the absorption of the electromagnetic radiation to the wavelength region to be absorbed in a defined manner via the particle size. The silicon-containing particles can be used for biocompatible and biodegradable UV protection in cosmetic or medical formulations, such as preferably a sunscreen, or else in a cosmetic formulation for the UV protection of hair.

### BACKGROUND OF THE INVENTION

1. Field of the Invention

The invention relates to the use of silicon-containing particles for protection of human cells from electromagnetic radiation in the UV range and optionally in the visible as far as the IR range, where the particles preferably take the form of clusters of primary particles having a particle size in the range from 5 to 100 nm. A particular advantage of the use according to the invention is the possibility of matching the absorption of the electromagnetic radiation to the wavelength region to be absorbed in a defined manner via the particle size. The silicon-containing particles, especially particles consisting of silicon, can be used as biocompatible and biodegradable UV protection in cosmetic or medical formulations, such as preferably a sunscreen, or else in a cosmetic formulation for the UV protection of hair.

2. Discussion of the Background

Known UV protection formulations are based on UV filters of different concentration. The UV filters used may act by different mechanisms. The known UV filters have a typical absorption spectrum and have to be combined with other UV filters since they are usually not active over the entire spectrum. Thus, the sun creams are frequently based on mixtures of different UV filters. In the sun creams, the UV filters work by coating the skin and are partly absorbed into the horny layer of the skin.

Standard UV filters, such as organic UV filters, work by a Stokes shift in the absorbed light to the longer-wave wavelength range. The UV filters are often classified in terms of their absorption spectrum as UVA, UVB and broadband filters (UVA/UVB filters). Organic UV filters are often derivatives of, for example, camphor or salicylic acid. Additionally known are inorganic UV filters such as fine titanium dioxide and zinc oxide particles. An advantage and simultaneously a disadvantage of these particles is their visibility. On the one hand, it is possible to check application via visibility, and so these UV filters are generally employed in sunscreen products for children. On the other hand, acceptance of visible UV filters in products for adults is very contentious.

A further disadvantage of inorganic UV filters such as zinc oxide is frequently the low particle size, which can enable penetration into the skin. Small amounts of the zinc particles have been detected in the blood and in the urine. For instance, no nanoparticulate zinc oxide-based sunscreen products are approved in Switzerland, and in Germany too the concentration must not exceed a fixed value of nanoparticulate zinc oxide.

### SUMMARY OF THE INVENTION

There is therefore a need for novel UV filters which do not have the aforementioned disadvantages. Preferably, the UV filters should be based on an inorganic material. It is further preferable when the UV filters do not decompose as a result of the action of UV radiation and do not form any bioincompatible breakdown or degradation products. In addition, the UV filters should not be absorbed or be degradable in the organism in the event of absorption.

Furthermore, silanes, especially silanes of high purity, constitute an important product class in many fields of use, such as the semiconductor industry or fibre optics industry. In the cosmetics industry, substantially only silicone oils or siloxanes are known as additives. These fields of use place high demands on the purity of these substances, especially in relation to trace contaminations and compatibility on the skin.

In the field of cosmetics, and here especially in the field of sun protection, a particularly adverse effect is associated with allergenic compounds, for example nickel or other production-related catalysts, or with TiO₂, which can have photocatalytic action. Contaminations of this kind, even in the lower % range and even down to the ppm (by weight) range, may be extremely troublesome in cosmetics applications.

The problem addressed was therefore that of developing a new UV filter. The new UV filter is not to decompose through UV radiation and is preferably to be biodegradable or degradable in the organism. In addition, the new UV filter is to be producible by an environmentally friendly and energetically improved process. Advantageously, the use of catalysts is to be dispensed with. In addition, the number of processing steps is to be reduced. A further problem addressed was that of finding a process which allows high throughputs of the compounds to be produced in a continuous or batchwise manner.

The present invention relates to a method for protection of a cell from electromagnetic radiation, the method comprising:
contacting the cell with a silicon-containing particle to absorb said electromagnetic radiation;
wherein said electromagnetic radiation has a wavelength range of from 10 to 1500 nm.

In addition, the present invention relates to a method for increasing the refractive index of a cosmetic, dental, medical or pharmaceutical formulation, said method comprising:
contacting the cosmetic, dental, medical or pharmaceutical formulation with a silicon-containing particle which absorbs electromagnetic radiation which has a wavelength range of from 10 to 1500 nm.

The present invention further relates to a biocompatible UV protection composition and/or biodegradable UV protection composition, comprising:

silicon-containing particles, comprising primary particles of 5 to 100 nm and optionally clusters of these primary particles, having a silicon content of greater than or equal to 90% by weight to 100% by weight;

wherein said particles absorb electromagnetic radiation in the UV range.

Moreover, the present invention relates to a process for producing silicon-containing particles, said process comprising:
a) decomposing at least one gaseous silicon compound or one which is gaseous at elevated temperature,
b) optionally in the presence of at least one gas which is reactive under the reaction conditions or of a mixture of reactive gases,
c) in the presence of a diluent gas in an essentially oxygen-free atmosphere under (i) thermal conditions and/or (ii) in a plasma, and
d) effecting deposition with a fluid as silicon-containing particles.

### BRIEF DESCRIPTION OF THE DRAWINGS

FIG. 1a shows a TEM image of the Si particles (Si) produced.

FIG. 1b shows the UV-vis spectrum of the product sample 4 (P4), silicon particles of size 10-50 nm with median about 35 nm, and the product 3 (P3) silicon particles of size 10-50 nm with median about 25 nm.

FIG. 2: shows the refractive index of silicon as a function of wavelength (λ), spectrum e: refractive index of silicon, line f: refractive index n=4.

FIG. 3a shows the silicon-containing particles according to Working Example 2a.

FIG. 3b shows silicon-containing particles according to Working Example 2b.

### DETAILED DESCRIPTION OF THE INVENTION

Any ranges mentioned herein-below include all values and subvalues between the upper and lower limits of the range.

Completely surprisingly and unexpectedly, it was possible to solve the above-mentioned problems by treating a silane mass flow in a plasma-thermal manner. The silane mass flow, preferably comprising monosilane SiH₄, is sent to a plasma discharge arrangement together with a contamination which, in accordance with the invention, comprises silicon-halogen compounds, for example chlorine-, bromine-containing compounds, and reacted therein, by way of example in a high-voltage pulse discharge.

The new process approach is to treat the mass flow such that it can be characterized by a particle size distribution which can be used to adjust the spectral absorption properties in the UV range and in the visible range, and optionally in the IR range. With regard to the effect of the gas discharge treatment, it is assumed that the kinetics are promoted when silicon-H or Si—Cl radicals are excited by plasma-chemical means and these selectively and preferentially form silicon particles smaller than 100 nm, which results in accordance with the invention in a shift in absorption from the UV-C (100-280 nm) through UV-B (280 to 315 nm) to the UV-A (315 to 380 nm). As a function of the median primary particle size, the absorption in the UV-C and UV-B range can be enhanced compared to the absorption in the UV-A range.

In the process according to the invention, the mass flow is amenable to a simple workup, such as filtration and/or washing, by means of which the pure product is preferentially drawn off The residual gas obtained, which also contains traces of HCl as well as H₂, is recycled. For the known principles of gas discharge and plasma chemistry, reference is made to the relevant specialist literature, for example by A. T. Bell in “Fundamentals of Plasma Chemistry”, ed. J. R. Hollahan and A. T. Bell, Wiley, New York (1974).

The problems were likewise solved in a surprising manner by converting silicon-containing particles by conversion of monosilane or higher H-silanes, such as those of the formula MeHₙ or Me₂H₂ₙ₋₂, where Me is silicon and n is an integer, halosilanes, higher halosilanes or alkoxysilanes, optionally with C- or N- or Ge-containing species, in a thermal process, optionally combined with a plasma process, or in a pure plasma process. Higher H-silanes or chlorosilanes are also referred to as polysilanes or polychlorosilanes. The higher H-silanes or higher chlorosilanes are converted to the gas phase prior to the conversion thereof. The halogen may be selected from fluorine, chlorine, bromine and iodine, and is preferably chlorine.

According to the invention, the problems are likewise solved by the inventive use of silicon-containing particles, especially of amorphous particles containing pure silicon, by using the particles for protection of cells from electromagnetic radiation in the wavelength range from 10 to 2500 nm, especially to 1500 nm. More particularly, the particles are used for protection of cells from damage by UV radiation. The protected cells include human, animal and/or else plant cells, preference being given to protecting epidermal cells such as skin cells, horny layer skin cells or else hair cells from being affected by electromagnetic radiation. The cells need not necessarily come into contact with the particles, but can also be protected indirectly by using the particles, for example, on lenses of sunglasses or for modification of textile materials, or in connection with scientific or industrial applications which serve for protection of cells, for example of bacteria cultures.

Preferred pure silicon-containing particles, especially primary particles, which are preferably present in clusters, have a silicon content of greater than or equal to 50% by weight to 100% by weight in relation to the overall composition of silicon-containing particles. More particularly, the silicon content is greater than or equal to 55% by weight, the silicon content preferably being greater than 80% by weight, 90% by weight, 95% by weight, 98% by weight, 99% by weight, 99.5% by weight, 99.99% by weight, 99.999% by weight to 100.0% by weight, and the particles optionally have, based on the silicon content in % by weight, a content of carbon and/or oxygen, where the particles are preferably essentially amorphous. The oxygen content is less than 50% by weight, preferably less than 10% by weight, more preferably less than 1% by weight. Particular preference is given to pure silicon-containing particles, especially primary particles, which are preferably present in clusters, having a silicon content of greater than or equal to 90% by weight to 100% by weight in relation to the overall composition of silicon-containing particles. In addition, silicon-containing particles may comprise from 0% to 10% by weight of selenium as free-radical scavenger. Preferably, the particles may comprise 0.0001% to 5% by weight of selenium.

The invention likewise provides for the use of the silicon-containing particles a) for absorption of electromagnetic radiation in the wavelength range of greater than or equal to 10 nm to 1100 nm, especially in the wavelength range from 10 nm to 450 nm. Particular preference is given to using the silicon-containing particles as UV protection from electromagnetic radiation, preferably as UV protection from 180 to 400 nm, more preferably from 200 to 380 or to 400 nm. It is likewise preferable that the silicon-containing particles can also protect cells from electromagnetic radiation in the wavelength range of extreme UV such as 10 to 100 nm or to 120 nm, in the far UV from 200 to 280 nm, in the middle UV from 280 to 315 nm and/or in the near UV from 315 to 380 nm, and, according to the size of the primary particles, from 400 to 750 nm in the visible range and optionally in the IR range above 750 nm to about 1500 nm. The defined absorption in the aforementioned ranges can be set specifically via the content of the respective median primary particle sizes.

It has been found that the spectroscopic properties of the amorphous particles can also be adjusted directly via the production process in terms of the size distribution of the primary particles and/or cluster formation, and optionally via an addition of carbon, nitrogen, germanium or further silanes.

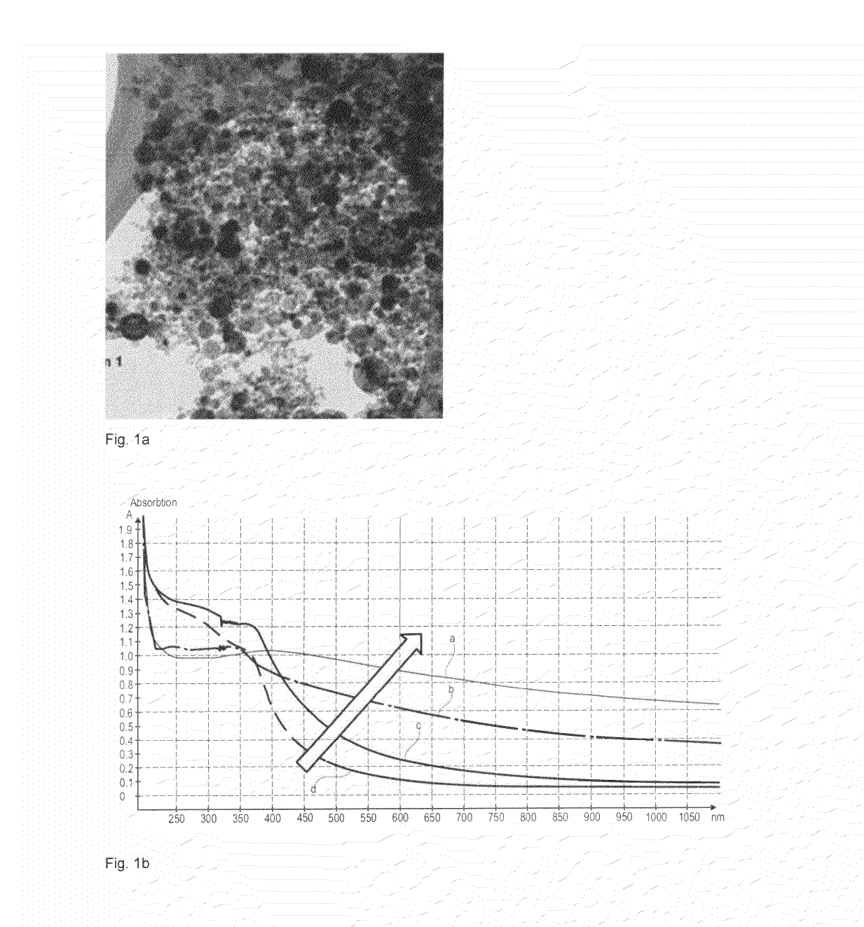

It was thus possible to directly adjust the absorption via the particle size of the silicon-containing particles, the primary particles and clusters of primary particles. Surprisingly, particles having a primary particle size of 5 to 80 nm and different median particle sizes d₅₀ around 10 nm, 15, 20, 25, 30, 35 and 40 nm have distinctly different absorption in the UV and visible ranges. Silicon particles having a median primary particle size d₅₀ of 15 to 30 nm, preferably 25 nm to 30 nm, especially with d₉₀ of 3 to 50 nm, absorb much more strongly in the UV region than in the visible region, whereas particles having a median particle size of the primary particles of 35 to 40 nm, especially with d₉₀ of 10 to 50 nm, absorb with comparable intensity in the UV/vis range (FIG. 1b).

Particular preference is given to the use of silicon-containing particles that are essentially amorphous particles. Amorphous particles are considered to be those having a crystallinity of less than or equal to 2%. Preferably, the amorphous silicon-containing particles have a median primary particle size d₅₀ of 20 nm, 25 nm, 30 nm or 40 nm, preferably in each case independently with a low scatter of +/−10 nm, especially +/−5 nm, for d₉₀.

The clusters of the primary particles may be 30 to 400 nm in size, the clusters having typical mean sizes around 150 nm plus/minus 50 nm or alternatively of 50 to 100 nm.

Inventive silicon-containing particles having primary particles having a median primary particle size of 10 to 30 nm, such as preferably around d₅₀=13.2 nm and preferably d₂₀=7.2 nm and d₉₀=23.5 nm, have a transparency of less than 40% (absorption greater than or equal to 0.6) in the wavelength range from 180 to 1000 nm, especially a transparency of less than or equal to 20% (absorption greater than or equal to 0.8) at 180 to 700 nm, and preferably additionally a transparency of less than or equal to 5% (absorption greater than 0.95) from 180 to 475 nm, the primary particles preferably forming clusters of greater than or equal to 50 nm and being amorphous.

Alternative inventive silicon-containing particles having primary particles having a median primary particle size of 35 to 40 nm have a transparency of less than 40%, meaning an absorption greater than or equal to 0.6, in the wavelength range from 180 to 400 nm, especially a transparency of less than or equal to 0% (absorption greater than or equal to 1.0) at 180 to 350 nm, and preferably additionally a transparency of less than or equal to 0% (absorption greater than 1.2) from 180 to 300 nm, the primary particles preferably forming average clusters of 30 to 400 nm, especially around 150 nm plus/minus 50 nm, and being amorphous.

The invention further provides for the use of silicon-containing particles having a content of greater than or equal to 40% by weight, especially greater than or equal to 50% to 100% by weight, of silicon and a particle size, especially a primary particle size, of 1 to 500 nm. Preferably, the silicon content in the overall composition of the silicon-containing particles or in the respective individual particles is 70% by weight, such as SiC, to 100% by weight, especially 80% to 100% by weight, preferably 90% to 100% by weight, more preferably 99.5% to 100% by weight, of silicon, and optionally additionally at least carbon and/or oxygen. It is likewise possible to use silicon-containing particles consisting essentially of amorphous pure silicon particles or else of amorphous pure silicon carbide, silicon nitride, the silicon-germanium particles of the aforementioned primary particle size or mixtures of these.

The invention provides a large-scale industrial process, preferably a continuous process, for preparing silicon-containing particles, especially comprising primary particles and optionally clusters of primary particles.

The reaction, especially comprising the breakdown and formation of the particles, can be effected at temperatures of 150° C. upwards, preferably from 400 to 1500° C., for production of amorphous powders. For production of amorphous particles, short contact times, preferably at temperatures below 1300° C., are chosen. Alternatively, the formation of amorphous primary particles can be effected at temperatures around 1300° C., preferably less than or equal to 1100° C. The particles are deposited in a cooler zone of the reactor. Preferred contact times are from 10 to 600 milliseconds.

For instance, customary conventional processes based on a conversion of SiCl4 require 30 kW/kg or more. The processes according to the invention are preferably based on a reaction of monosilane with an energy requirement of less than 10 kW/kg, more preferably around 5 kW/kg. In addition, for the conversion in the (cold) plasma, the energy requirement is reduced further to less than 4 kW/kg.

Particular preference is given to silicon-containing particles in which less than 20% of the particles have a deviation from the median particle size d₅₀ and less than 5% have a deviation of greater than or equal to 50% from the median particle size d₅₀.

Preference is further given to silicon-containing particles wherein the primary particles have a median diameter d₅₀ (determined by TEM evaluation; TEM=transmission electron microscopy) in the range from 5 to 80 nm, preferably from 20 to 50 nm, and which preferably take the form of aggregated clusters. The clusters can also be referred to as agglomerates, a cluster in the present case being understood to mean aggregated or fused primary particles. For instance, the primary particles can form clusters in which at least two primary particles are fused to one another at their surfaces. These clusters may take the form of linear chains or of wires, or else be in branched form.

Whether the particles are spherical or take the form of whiskers depends on factors including the H₂ concentration in the preparation. According to the temperature profile, purity (presence of metallic elements, for example iron (Fe) in the gas stream), diluent gas (concentration, flow rate), production conditions, it is possible to isolate primary particles or to obtain predominantly primary particles agglomerated to clusters. For instance, in the case of a dilute process regime, it is possible to isolate predominantly primary particles, and, in the case of high process gas concentration and/or high temperature, for example at 1500° C., to isolate clusters.

The invention also provides for the use of clusters of the primary particles as protection from electromagnetic radiation. As well as UV-VIS-IR, this includes the adjoining terahertz and the high- and ultra-high-frequency area, and the range of radio waves up to long waves having frequencies above 1 hertz.

According to a further alternative, the silicon-containing particles preferably comprise primary particles that are essentially spherical. The mean sphericity, defined as the aspect ratio of the diameters at a 90° angle to one another, is preferably less than or equal to 1.6, preferably less than or equal to 1.4 to greater than or equal to 0.9, preferably from 0.95 to 1.2. The aspect ratio close to the spherical form allows an ideal correlation of the UV absorption to the primary particle size and good homogenizability.

In addition, the silicon-containing particles may comprise primary particles and clusters of primary particles; more particularly, the clusters have a size of 10 nm to 3 μm, preferably of 100 nm to 3 μm, further preferably of 1 μm up to 6 μm. Preferred silicon-containing particles comprise silicon (Si) or optionally SiC, SiGe, SiN compounds of greater than or equal to 99.9999% by weight of silicon. Optionally, the SiC, SiGe, SiN compound has a corresponding content of these compound elements. Preferably, the silicon content is greater than or equal to 94.99% by weight, more preferably greater than or equal to 97.999% by weight. Likewise preferably, the silicon content may be 98.99% to greater than or equal to 99.98% by weight. Likewise preferred are particles comprising silicon-nitrogen, silicon-carbon and/or silicon-germanium compounds, such as silicon nitride, silicon carbide, silicon carbide in a silicon matrix. The silicon-nitrogen, silicon-germanium, silicon-carbon compounds may also be present in particulate form in a matrix of essentially pure silicon. Preferably, the silicon-containing particles are present essentially without any outer matrix or coating which may also include a passivating oxide layer. In one alternative, the silicon-containing particles are present with a silicon dioxide zone, the so-called core-shell, of typically 1 nm. Preferably, or in the ideal case, the zone comprises a monolayer on the surface.

It is particularly preferable in accordance with the invention when the silicon-containing particles are of high purity, especially of ultrahigh purity. The particles are considered to have high purity when silicon having a content greater than or equal to 99.99% by weight is present in the overall composition, preferably with a content of greater than or equal to 99.999% by weight. Ultrahigh-purity silicon-containing particles are considered to be those having a content of greater than or equal to 99.9999% by weight of silicon in the overall composition, in which case a silicon dioxide core-shell is not an option.

The impurities in the respective reactants and process products are determined by means of sample digestion methods known to those skilled in the art, for example by detection in ICP-MS (analysis for the determination of trace impurities).

In the inventive particles, the primary particles and optionally the clusters, depending on their size, have a content of less than or equal to 2 atom %/cm³ in the overall composition, preferably less than or equal to 2000 ppm (by weight) of oxygen, preferably less than 1000 ppm (by weight), further preferably less than 10 ppm (by weight).

The analysis is effected, in addition to ICP-MS and HP-GD-MS (high purityglow discharge mass spectroscopy), preferably by means of neutron activation analysis (NAA). NAA is a highly sensitive physical technique in analytical chemistry for qualitative and quantitative trace analysis, in which the sample to be analysed is bombarded with neutrons or other uncharged particles.

In NAA, a sample of only a few milligrams to, as the case may be, a few μg is exposed to the neutron stream, for example from a nuclear reactor. The neutrons react with the nuclei of the sample and convert the stable isotopes to radioactive isotopes having a mass number one higher than the mass number of the stable isotope. In this first nuclear process, a prompt γ quantum is emitted, the energy of which can be measured and which gives information about the original nucleus. In most studies, however, it is the breakdown of the radioactive nucleus formed that is used for analysis. It subsequently breaks down with its typical half-life, emitting a beta particle and characteristic gamma radiation which is analysed in a gamma spectrometer. In this way, virtually all elements that occur in a sample are detectable quantitatively and qualitatively. The known properties of the atomic nuclei can be used to determine not only the content of elements but even their isotopes. The measurements can be conducted, for example, at the Berlin Neutron Scattering Center (BENSC).

The content of diluent gases, such as xenon, argon, krypton, or else nitrogen, in the overall composition of the particles is less than 1% atoms/cm³, especially less than or equal to 1000 ppm (by weight), 10 ppm (by weight), 1 ppb (by weight) down to the detection limit, for example to 1 ppt (by weight).

The detection limits for the determination of xenon (Xe), argon (Ar), krypton (Kr), or else nitrogen, by neutron activation with an irradiation time of 1 hour, at a so-called flux density of thermal neutrons of 10¹⁴ cm⁻² s⁻¹ are about 10⁻⁸ g (Ne, Xe), about 10⁻⁹ (Kr), about 10⁻¹⁰ g (Ar) and about 10⁻⁵ g for oxygen (O₂). The detection limit, especially for small samples, may thus be less than or equal to 1000 ppm (by weight), 10 ppm (by weight), 1 ppb (by weight) or down to the detection limit, for example to 1 ppt (by weight).

To produce the particles containing high- or ultrahigh-purity silicon, a high- to ultrahigh-purity silane is used, for which the definition of pure silane to ultrahigh-purity silane of semiconductor quality is used, such as monomeric and/or polymeric monosilane, H-silane and/or chlorosilane, especially of the general formula I, II and/or III, or a mixture of the comprising, such as ultrahigh-purity tetrachlorosilane, ultrahigh-purity trichlorosilane and/or ultrahigh-purity dichlorosilane, preferably having a silane content of 80% to 99.9999999% (by weight), ad 100% (by weight) optionally polysilanes, and with a total contamination of less than or equal to 100 ppm (by weight) to 0.001 ppt (by weight) as high-purity silane, preferably less than 50 ppm (by weight) to 0.001 ppt (by weight) as ultrahigh-purity silane, preferably less than or equal to 40 ppm (by weight) to 0.001 ppt (by weight) of total contamination with the elements specified hereinafter. Alternatively, rather than a single silane, it is also possible to use a mixture of silanes, provided that it meets the aforementioned profile of requirements on the silane content.

The invention also provides for the use of silicon-containing particles comprising
a) primary particles of primary particle size from 1 nm to 500 nm, especially having primary particles of 3 to 100 nm, especially 5 to 500 nm, 5 to 100 nm, preferably from 5 to 80 nm. These primary particle sizes are preferably obtained via a plasma method. It is additionally preferable when the particles simultaneously have
b) a median primary particle size d₅₀=16 nm, 20 nm, 25 nm, 30 nm or 40 nm, especially each independently with a low scatter of +/−5 nm for d₉₀, where at least a portion of the primary particles is preferably present in the form of clusters. Alternatively, primary particles have a particle size of 10 to 50 nm +/−35 nm, especially primary particles of 10 to 50 nm +/−25 nm. Primary particles obtainable via an FSR process generally include primary particles having particle sizes of 100 to 350 nm, and preferably clusters of the primary particles.

Preferably, the inventive clusters have a size of 10 nm to 3 μm, further preferably of 100 nm to 3 μm, further preferably of 200 nm to 3 μm.

It is preferable when greater than or equal to 70% by weight of the primary particles are present as a cluster, preferably greater than 80% by weight, more preferably from 85% by weight to 100% by weight, further preferably greater than 90%, 95%, 98%, 99.5% by weight.

According to the primary particle size, the silicon-containing particles have a pale yellow, orange or light brown colour, and can therefore also be used if required as pigments in cosmetic products. The particularly surprising property is manifested here, namely that of simultaneous UV protection and skin-like colour. By virtue of the pleasant skin-like warm intrinsic colour, the silicon-containing particles applied to the skin and/or the lips are not perceived as unappealing and unpleasant, as is the case for the known white UV filters.

The inventive silicon-containing particles may also have a BET surface area of more than 10 m²/g, preferably greater than or equal to 100 m²/g.

According to a particularly preferred embodiment, silicon-containing particles are used which are especially amorphous, where the particles have the following characteristics of electromagnetic radiation absorption:
a) with a primary particle size of 10 to 50 nm, especially with a median particle size d₅₀ around 35 nm, the absorption in the wavelength range from 250 to 400 nm as compared with the absorption in the wavelength range from 400 to 750 nm is equal with a deviation of +/−40%, especially preferably +/−30%, preferably +/−20%, more preferably +/−10%, and/or
b) with a primary particle size of 10 to 50 nm, especially with a median particle size d₅₀ around 25 nm, the ratio of the absorption in the wavelength range from (i) 250 to 400 nm as compared with the absorption at a wavelength of (ii) 550 nm is about (i):(ii)=2:1 to 8:1, especially 2:1 to 6:1, preferably 3:1 to 6:1, with a deviation of +/−40%, preferably +/−30%, preferably +/−20%, more preferably +/−10%, and/or
c) with a primary particle size of 10 to 50 nm, especially with a median particle size d₅₀ around 25 nm, the ratio of the absorption in the wavelength range from (i) 250 to 400 nm as compared with the absorption at a wavelength of (ii) 500 nm is about (i):(ii)=1.5:1 to 8:1, especially 1.8:1 to 5:1, preferably from 2:1 to 4:1, with a deviation of +/−30%, especially preferably +/−20%, more preferably +/−10%, and/or
d) with a primary particle size of 10 to 50 nm, especially with a median particle size d₅₀ around 25 nm, the ratio of the absorption in the wavelength range from (i) 250 to 350 nm as compared with the absorption at a wavelength of (ii) 450 run is about (i):(ii)=2:1 to 4:1 with a deviation of especially +/−30%, preferably +/−20%, more preferably +/−10%, and/or
e) with a primary particle size of 10 to 50 nm, especially with a median primary particle size d₅₀ around 25 nm, the ratio of the absorption at a wavelength of (i) 250 nm as compared with the absorption at a wavelength of (ii) 450 nm is about (i):(ii)=2:1 to 4:1 with a deviation of especially +/−30%, preferably +/−20%, more preferably +/−10%, and/or
f) with a primary particle size of 10 to 50 nm, especially with a median particle size d₅₀ around 25 nm, the ratio of the absorption at a wavelength of (i) 350 nm as compared with the absorption at a wavelength of (ii) 450 nm is about (i):(ii)=2:1 to 3:1 with a deviation of especially +/−30%, preferably +/−20%, more preferably +/−10%.

The values specified in a) to f) were achieved especially for pure, high-purity to ultrapure and essentially amorphous silicon-containing particles having a silicon content of greater than or equal to 98% by weight and optionally having a content of carbon and/or oxygen. Preference is given to a silicon content of greater than or equal to 99.5% by weight and optionally additionally oxygen, based on 100% by weight in the overall composition. Preferably, the absorptions specified in each case are averaged over the wavelength range. The invention likewise provides for the use of silicon-containing particles, especially amorphous and essentially silicon-containing particles of primary particle size from 1 to 100 nm, for increasing the refractive index of a cosmetic, dental, medical or pharmaceutical formulation. A conceivable medical formulation is a spray bandage or a wound dressing.

The invention likewise provides for the use of silicon-containing particles selected from particles having a content of pure silicon and SiC with silicon 70% to 90% by weight and 10% to 30% by weight of carbon and pure SiC for increasing the refractive index of a formulation or a material. Preferably, the silicon-containing particles include silicon to an extent of 60% to 100% by weight and are made up to 100% by weight, for example to an extent of 0% to 40% by weight, by a content of carbon and optionally oxygen, the particles preferably being transparent. Further preferably, the silicon particles comprise silicon to an extent of 70% by weight and are made up to 100% by weight by a content of carbon and optionally oxygen. In this case, silicon-containing particles, especially selected from particles having a silicon content greater than 90% to 100% by weight, preferably greater than or equal to 91% to 100% by weight, preferably greater than or equal to 95%, 98%, 99.5% to 100% by weight, have a refractive index (n) of greater than or equal to 3.0 at a wavelength of 500 to 2500 nm, and/or a refractive index (n) of greater than or equal to 4.0 at a wavelength of 200 to 500 nm, especially of 280 to 400 nm. Preferably, the particles, especially pure and amorphous silicon particles, have a refractive index (n) of greater than or equal to 5 to 7 in the wavelength range from about 280 to 400 nm, more preferably of 6 to 7. Preferably, the essentially pure, amorphous silicon particles having a median primary particle size of 20 to 40 nm have these refractive indices. The refractive indices thus allow quality control of the purity of the silicon-containing particles produced.

The invention likewise provides for the use of silicon-containing particles, especially selected from particles having a silicon content of greater than or equal to 50% by weight to 100% by weight, which may optionally comprise 0% to 50% by weight of carbon, preferably 0.001% to 30% by weight, preferably 0.001% to 20% by weight, more preferably 5% to 15% by weight, of carbon, the carbon content preferably being around 10% by weight of carbon with a range of variation of +/−3% by weight. Additionally or alternatively, the silicon-containing particles may include 0% to 20% by weight of oxygen. Preferably, the outer coating, meaning the core-shell, has an oxygen content. Preferably, the oxygen content is from 0.0001% to 10% by weight, further preferably from 0.0001% to 5% by weight. It is preferable when the median primary particle size is 20 to 40 nm and/or the primary particles have a size of 10 to 80 nm.

It is further preferable when the particles have a core-shell having an oxygen content, the particles in this case preferably having a content of Si—O and/or Si—OH groups. Particles having a core-shell, which can also be produced in a defined manner in the process, are amenable, for example, to a silanization and hence to a further modification and attachment or incorporation into other materials.

A further advantage of the inventive particles is the usability thereof as inorganic UV protection and preferably protection from scratches, for example in the field of polymeric or inorganic surfaces. Use as UV and scratch protection is not restricted to direct use for protection of cells, but can also be used indirectly for protection of cells, for example in sunglasses and the like, and serve indirectly for protection of cells.

In addition, in one alternative, the silicon-containing particles present in the form of primary particles and optionally clusters of the primary particles, and optionally containing silicon carbide, silicon nitride, silicon-germanium, may additionally be doped with an electron acceptor or electron donor. For doping, during the production, at least one doping gas reactive under the reaction conditions is added. A preferred example is diborane. It is likewise possible to add a selenium-containing gas.

In a further preferred alternative, the invention provides for the use of silicon-containing particles comprising primary particles of 1 to 100 nm, preferably 5 to 100 nm, and optionally clusters of these primary particles, having a silicon content of greater than or equal to 90% by weight to 100% by weight, as biocompatible UV protection and/or biodegradable UV protection. It is further preferable when the particles can be degraded by cells such as the skin cells or body cells. For instance, the body can preferably absorb and degrade the amorphous silicon particles. The silicon particle is converted gradually to SiO₂ in the aqueous biological system. Depending on the pH of the biosystem, it is dissolved.

The particles may be present essentially as spherical particles or else as platelet-shaped particles having a layer thickness of 1 to 80 nm, especially 20 to 45 nm. The particles may be present as platelet-shaped particles when the particles are deposited on a cold surface or particles are introduced into a film.

The invention likewise provides a process for producing silicon-containing particles, and silicon-containing particles obtainable by this process, by
a) decomposing at least one gaseous silicon compound or one which is gaseous at elevated temperature,
b) optionally in the presence of at least one gas which is reactive under the reaction conditions or of a mixture of reactive gases, especially at elevated temperature or in the plasma-reactive gas,
c) in the presence of a diluent gas in an essentially oxygen-free atmosphere under (i) thermal conditions and/or (ii) in a plasma, and
d) effecting deposition with a fluid as silicon-containing particles.

The silicon compound may be a gaseous compound such as preferably monosilane or else a compound which is converted to the gas phase at elevated temperature and/or reduced pressure.

Deposition in the form of silicon-containing particles, especially of the solid formed, can be effected in a fluid, for example by scrubbing, preferably by cooling to quenching the decomposition products of the silicon compound and optionally of the at least one reactive gas with a liquid or gaseous fluid, such as paraffin, N₂, noble gases, CO₂, gaseous or supercritical CO₂. Preferably, the decomposition products can be deposited and especially isolated as silicon-containing particles with a fluid in the form of a solution, emulsion, suspension, gel, foam, aerosol or smoke. The particles thus produced, according to the process conditions, may be deposited as predominantly isolated primary particles which preferably need not be stabilized any further, or as predominantly clusters of primary particles, each in amorphous form. Stabilization can be improved, for example, by using what are called ionic liquids.

Further examples of useful fluids include: natural or synthetic oils/fats, emulsifiers, waxes, hydrocarbons such as paraffin oil, silicone oil, vegetable fats and oils, synthetic glycerides, cocoa butter, almond oil, peanut oil, shea butter, animal waxes (wool wax, beeswax), hydrogenated oils (hydrogenated castor oil, hydrogenated soya oil), emulsifiers, either of the O/W type (e.g. polysorbate, macrogol ethers, fatty alcohol sulphates) or of the W/O type (wool wax alcohols, sorbitan fatty acid esters, monoglycerides), (partly) oxidized hydrocarbons, fats/oils, etc., and further customary and preferably pharmacologically compatible fluids known to those skilled in the art.

Useful gaseous silicon compounds generally include all hydrogen-containing silanes such as monosilane, disilane, trisilane and mixtures comprising at least one of the silanes mentioned, and also halogen- and hydrogen-containing or purely halogen-containing silanes and polysilanes, which may also be used in the process in the mixture. Preferably, the silicon compound may include traces of Si—Cl, Si—Br and/or halosilanes, or a Si—Cl-containing compound added or present in traces. The silicon compound which is gaseous at elevated temperature may also include hydrocarbon-containing silanes. Preferred silanes are halosilanes, chlorosilanes such as dichlorosilane, trichlorosilane, tetrachlorosilane, hexachlorodisilane, methyltrichlorosilane, polyhalosilanes, and pure H-silanes such as monosilane, hydrogen-containing polysilanes or polyhalosilanes and/or at least one alkoxysilane.

Particularly preferred cooling conditions are elucidated in detail hereinafter. As detailed above, the powders can be formed in two ways. For instance, the cooling and formation of the silicon-containing particles can be effected by reaction in the cool reactive gas stream, especially cooled with liquid nitrogen. The reactive gas stream may comprise hydrocarbons. After the silicon-containing particles have been formed, the breakdown products are cooled further by introducing the gaseous decomposition products into fluids, coolants, for example liquid helium, or into a liquid reactive gas. In this case, the reactive gas binds directly to the silicon-containing particle. This can give rise to a functionalization.

Cooling of the process gas from the hot non-thermal plasma can also be effected by a suitable process regime, for example by introducing it into inert, cool and readily evaporable media or by introducing it into liquid silicon compounds and/or boron- or selenium-containing compounds. Generally, the deposition can be effected by virtue of the fluids for deposition having a temperature well below 1000° C., preferably below 200° C., further preferably below 100° C., more preferably below 50° C. to −273° C. Particular preference is given to rapid deposition by establishing a temperature differential of greater than or equal to 100° C., especially greater than or equal to 200° C., preferably of greater than or equal to 500° C., within one minute, preferably within 1000 milliseconds, below the respective melting point of the silicon-containing particles, more preferably within less than or equal to 200 milliseconds, in order to obtain essentially amorphous particles.

Alternatively, the amorphous primary particles can be obtained in a plasma present in a non-thermal equilibrium, the temperatures of which are less than or equal to 1050° C., preferably less than or equal to 700° C., more preferably less than or equal to 150° C. In another variant, the process is preferably conducted in the low-temperature range, i.e. in the range between 373 Kelvin and greater than 0 Kelvin.

Preferably in accordance with the invention, the plasma comprises the conditions of a gas discharge, especially the non-thermal plasma.

Non-thermal plasmas used in accordance with the invention are produced, for example, by a gas discharge or by incidence of electromagnetic energy, such as by incidence of radio waves or microwaves, in a reaction chamber. The plasma is thus produced not by high temperatures as in the case of thermal plasmas, but by non-thermal ionization processes. The person skilled in the art is aware of such plasmas. In this regard, what is called the Penning ionization process is cited by way of example.

For the processes detailed above, gas discharges conducted in a non-thermal equilibrium were used. Non-thermal in the sense of the invention means that the electrons as energy-imparting species have a higher temperature and hence a higher energy than the heavier charged or uncharged particles (for example N, N₂, N₂⁺, N⁺, Si, SiH, SiH₂⁺, C, H, H₂, NH). The energies required for the purpose can be produced by means of power supply units known or familiar to those skilled in the art. The nonthermal plasma generally has electrons having an energy in the range from 0.1 to 100 eV, preferably from 1 to 50 eV. The heavier particles have an energy in the range from 0.000 001 to 10 eV, preferably from 0.01 to 0.1 eV.

It has been found that, surprisingly, such a non-thermal gas discharge can advantageously be produced by dimming (phase gating control) or/and by means of pulsewidth modulation or via the pulse frequency, in which case the electrodes are advantageously designed as hollow electrodes with preferably porous end faces, for example made from sintered metal, by virtue of a two-dimensional parabolic shape. Thus, the gas stream is distributed homogeneously over the electrode surface. The two-dimensional mushroom-like surface can be described by the following relationship:

F(r)=r², with 0.1<r<1.1 cm

The process according to the invention is generally conducted in non-thermal plasmas having temperatures of 100 to 3400° C., preferably of 700 to 999° C.

The plasma may be pulsed. Preferably, an essentially cylindrical plasma, which is especially non-thermal, is provided in a cylindrical region of the reaction cylinder.

In the syntheses in a plasma, it is appropriately possible to work with an inert gas, for example a noble gas or a mixture of noble gases, for example argon with small proportions of helium, and/or krypton, xenon, and also with reactive gases such as nitrogen, hydrogen, methane, carbon tetrachloride, etc. Other gas mixtures are known to those skilled in the art or can be found in relevant textbooks.

A further preferred embodiment of the process according to the invention includes the introduction of noble gas or of noble gas mixtures or of noble gas/gas mixtures composed of the combination of argon and/or helium, krypton, xenon as diluent gas, or of mixtures of nobles gases and reactive gases such as nitrogen, hydrogen, methane, carbon tetrachloride, etc., into the non-thermal plasma especially having temperatures of less than or equal to 3000° C., preferably less than or equal to 1900° C.

In the process according to the invention, it is preferably possible to use further silicon compounds that are gaseous at elevated temperature. These include hydrogen- and/or halogen-containing silanes, such as H-silanes, halosilanes, and/or hydrocarbon-containing silanes such as methylsilane, methyltrichlorosilane, dimethyldichlorosilane, chlorosilanes, and pure, highly pure and especially ultrapure H-silanes such as monosilane, and/or chlorosilanes such as dichlorosilane, trichlorosilane, tetrachlorosilane, hexachlorodisilane, methyltrichlorosilane, polyhalosilanes, hydrogen-containing polysilanes comprising exclusively hydrogen or hydrogen and halogen. For production of the amorphous particles, it is likewise possible to use alkoxysilanes, preferably tetramethoxysilane, tetraethoxysilane or mixed tetraalkoxysilanes.

Silanes usable in accordance with the invention include silanes of the general formula I

HₓSiCl₄₋ₓ I

where x is independently selected from 0, 1, 2 and 3. Preferably, x is equal to 0, 1 or 2; preferably, x is equal to 0 or 1. More preferably, x is equal to 0, or a mixture comprising at least two monomeric chlorosilanes of the formula I, selected from tetrachlorosilane, trichlorosilane and dichlorosilane, preferably pure tetrachlorosilane or pure tetrachlorosilane having a content of trichlorosilane and/or dichlorosilane.

It is also possible with preference to use polyperchlorosilane mixtures comprising polyperchlorosilanes having 2 to 8 silicon atoms. Polychlorosilanes having up to 6 silicon atoms are readily evaporable, whereas compounds from 7 silicon atoms upwards are processed as aerosol. Particular preference is also given to the use of higher molecular weight polychlorosilanes having at least three silicon atoms, especially having 3 to 8 silicon atoms.

Preference is given to polyperchlorosilane mixtures comprising polyperchlorosilanes having 2 to 100 silicon atoms. Particular preference is also given to the use of higher molecular weight polychlorosilanes having at least three silicon atoms, preferably having 3 to 50 silicon atoms, which can be prepared by comproportionation reaction of [SiCl]ₙ, where the [SiCl]ₙ molecule is preferably present as a six-membered ring network with n=6, and multiples thereof. The analysis is effected in the infrared spectral region or by Si29 NMR.

The use of polychlorosilanes according to the invention encompasses the homologous series of the polyperchlorosilanes of the general formula II

SiₙCl₂ₙ₊₂ II

with n≧2, which form linear and/or branched chains, and the polyperchlorosilanes which form rings or polymers, where the polymers may also be branched and/or cyclic, and likewise having the idealized formula III

SiₙCl₂ₙ III

with n≧3, and also the silicon chlorides having a lower chlorine content of the idealized formula IV:

SiCl_{1.5}. IV

More preferably, polychlorosilanes are compounds of the general formula II with n≧2, preferably with 2≦n≦100, further preferably with 2≦n≦50, preferably each independently with n greater than or equal to 2, 3, 4, 5, 6, 7, 8, 9 or 10, preferably 2 to 8, more preferably with n equal to 2 or 3, where the compounds may form linear or else branched chains. Likewise preferred are compounds of the general formula III which form rings or polymers with SiₙCl₂ₙ, with n≧3, especially with 4≦n≦100, preferably 4≦n≦50, more preferably each independently with n≧4, n≧5, n≧6, n≧7, n≧8, n≧9 or n≧10, or else polychlorosilanes having a lower chlorine content and of the general formula IV with SiₙCl_{1.5n}, with n≧4 or n≧5, especially with 6≦n≦200, preferably with 8≦n≦100.

Particular preference is given to using a polychlorosilane (PCS), especially octachlorotrisilane or an octachlorotrisilane in a mixture with higher molecular weight polychlorosilanes, preferably polyperchlorosilanes, where the polychlorosilane has a content of octachlorotrisilane of 0.20% to 99.9999% by weight, preferably of 10% to 99.9999% by weight. These silanes may include the abovementioned impurities.

It is likewise possible to use a dissolved polysilane and/or polychlorosilane, for example in a reactive solvent, e.g. hydrocarbon as liquid, syrup, paste, cream, dispersion, emulsion, where the high-purity solvent is evaporated before the decomposition to give a reactive gas. In the context of the invention, reactive evaporable solvents are also considered to be reactive gases.

Preferably, an inert diluent gas is used in the process. According to the profile of requirements, it is possible to dispense with the diluent gas if a vacuum is applied at the same time. Preferred diluent gases are argon, helium, xenon, krypton or a mixture comprising at least two of the gases mentioned. Under the conditions mentioned, in the plasma or non-thermal plasma, nitrogen is not an inert diluent gas but a reactive gas. The diluent gas can preferably be mixed with the gaseous silicon compound and/or the reactive gas before being introduced into the reaction cylinder. Alternatively, the inert diluent gas is used for deposition of the particles. The breakdown of the silicon compound and optionally of the reactive gas can also be effected by transfer into a high-vacuum region. A high vacuum is considered to be a vacuum less than or equal to 0.01 bar, especially less than or equal to 0.001 bar, less than or equal to 0.0001 bar.

The pressure range is typically 0.001 mbar to 50 bar, preferably 1 mbar to 10 bar, further preferably 10 mbar to 5 bar. According to the desired breakdown and/or alloy and/or coating product, the process can also be effected within a pressure range from 1 to 50 bar, preferably at 2 to 50 bar, more preferably at 5 to 50 bar. This minimizes the formation of carbon-containing process gases. The person skilled in the art is aware that the pressure to be selected is a compromise between gas removal, agglomeration and reduction of the carbon-containing process gases.

Reactive gases usable with preference include nitrogen-containing compounds, germanium-containing compounds, hydrogen, nitrogen (N₂), NH₃, hydrazine, nitrogen-hydrogen acid, hydrocarbons such as methane, propane, butane, natural gas of ultrahigh purity, aromatic compounds such as toluene, especially oxygen-free compounds in each case, with the proviso that no water forms in the decomposition. Preferred reactive gases include hydrocarbons such as methane, ethane, propane, butane, mixtures of these gases, and also HCl and/or hydrocarbons with nitrogen.

For performance of the process, it is further preferred, in addition to the aforementioned features, when the gas discharge is a non-thermal plasma. Further preferably, the gas discharge is induced in a generator which also finds use in an ozonizer. For a definition of nonthermal plasma, reference is made to the relevant technical literature, for example to “Plasmatechnik: Grundlagen and Anwendungen—Eine Einführung [Plasma Technology: Fundamentals and Applications—An Introduction]; collective of authors, Carl Hanser Verlag, Munich/Vienna; 1984, ISBN 3446-13627-4”.

The specific power input is from 0.001 to 1000 W/cm².

More preferably, the specific energy input is from 0.1 to 100 Ws/cm². In these cases, the gap width (GAP) may be 1 mm. It is further preferable when the specific energy input is conducted by means of exact-phase instantaneous power measurement with a bandwidth of at least 250 kHz. The determination of the instantaneous power is effected in a standardized arrangement with discharge area 50 cm². The energy input to form the non-thermal plasma is preferably effected in such a way that very substantially homogeneous conditions are established in the plasma which forms for the conversion of the silanes and of other compounds containing C, N and/or Ge or the like. It is especially preferable here when the non-thermal plasma is operated at a voltage at which the discharge covers the entire electrode area.

According to a preferred alternative, the deposited particles are organofunctionalized at oxygen atoms and/or chlorine atoms present at the surface. Preferably, the particles are modified at the surface of the particles by reaction with a reactive organofunctional group of the silane. The modification can be effected via complexation or formation of covalent bonds. Generally, the silicon-containing particles can be modified at least partly with an organofunctional silane. Organofunctional silanes include silanes having unsaturated hydrocarbyl radicals, halogen-functionalized silanes such as preferably haloalkylsilanes such as monochlorotrimethylsilane, haloalkoxysilanes such as monochlorotrialkoxysilane, alkylenealkoxysilanes, alkylenehalosilanes, amino-functional silanes such as aminopropyltriethoxysilane, aminopropyltrialkylsilane, and organofunctional silanes. Organofunctional silanes also include organically functionalized silicon compounds and organosiloxanes.

The invention also provides formulations comprising the silicon-containing particles, where these formulations may comprise auxiliaries, solvents, emulsifiers and/or dispersants and/or suspension media. Particularly preferred formulations comprise amorphous silicon-containing particles having a content of 50% to 100% by weight of silicon, preferably comprising pure silicon particles having a silicon content greater than or equal to 99.95% by weight, more preferably greater than or equal to 99.9999% by weight, and having a median primary particle size d₅₀ of 5 to 500 nm. Preferably, d₅₀ is 5 to 40 nm, the particles preferably being in the form of a cluster of primary particles.

The invention also provides a formulation comprising silicon-containing particles as a cosmetic, medical or pharmaceutical formulation for UV protection of the skin or hair, especially a topical sunscreen formulation, for example a sunscreen cream, a sunscreen gel, sunscreen emulsion or oil, or a sprayable sunscreen formulation. Wound dressings and eye drops are other possible formulations. The formulation according to the invention preferably comprises at least one auxiliary such as bisabolol, wool waxes (lanolin), silicon dioxide, for example Aerosil®, bentonite, glycerol, cellulose ethers such as methyl cellulose, hydroxyethyl cellulose, ethyl hydroxyethyl cellulose, sodium carboxymethyl cellulose, alginate, starch, tragacanth, polyacrylic acids, polyvinyl alcohol, polyvinylpyrrolidone, vegetable or synthetic fats and oils.

A formulation according to the invention preferably comprises at least one auxiliary, additive or further customary formulation constituents. Preferably, the formulation is intended for topical use or for use in the eye, as a solution, suspension, emulsion. Preferred administration forms may include ointments, creams, hydrogels, gels, sprays, powders, film-forming sprays, films, and further cosmetic and/or medical formulations and administration forms that are familiar to those skilled in the art.

The particles according to the invention, because of their inertness and their transmission characteristics, are of particularly good suitability as UV protection compositions, since the particles according to the invention, such as the SiC powders, may be transparent and advantageously do not have any significant transmission at 300 nm.

The amorphous particles are preferably non-crystalline. An essentially amorphous powder is considered to be one which is x-ray-amorphous. An x-ray-amorphous powder is preferably considered to be a powder having a crystallinity of less than 2%. The crystallinity level is calculated by means of XRPD, employing the relationship:

Crystallinity in %=(100×A)/(A+B−C).

The parameters A, B and C mean:
A=total peak area of the reflections of the crystalline constituents of the diffractogram.
B=total area beneath the peak area A.
C=air scattering-, fluorescence- and instrument-dependent base area.

In the examples, the background area C was ascertained by reference to the XRD diagrams of the Si reference standard NIST 640 (Si standard=100% crystallinity). The area B corresponded to an inserted base profile and the constant base C. For the calculation, the “HighScore Plus Software” known to those skilled in the art was used.

In x-ray-amorphous powders, there are no sharp line forms in the XRPD, but only a few diffuse interferences at low diffraction angles. Substances having an x-ray diffraction diagram of this kind are referred to as x-ray-amorphous. A crystal, meaning an anisotropic, homogeneous body having a three-dimensional periodic arrangement of the sub-units, shows clearly defined resolvable reflections in the XRPD.

Both the particle sizes and the formation of clusters, for example agglomerates, and/or the presence of primary particles can be detected or determined, as well as other methods known to those skilled in the art, by means of screen analysis, transmission electron microscopy (TEM), scanning electron microscopy (SEM) or light microscopy.

Having generally described this invention, a further understanding can be obtained by reference to certain specific examples which are provided herein for purposes of illustration only, and are not intended to be limiting unless otherwise specified.

### EXAMPLES

### Working Examples

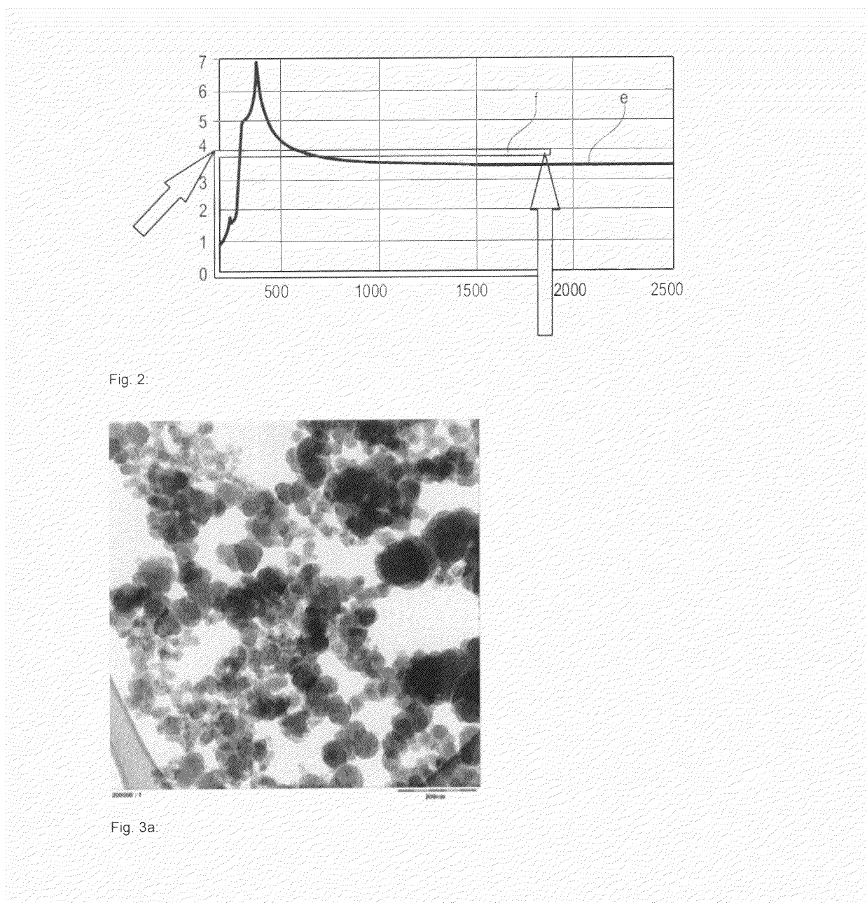

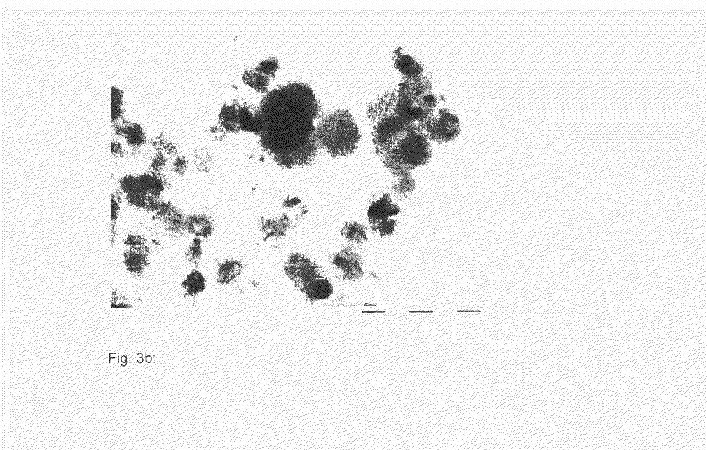

FIG. 1a shows a TEM image of the Si particles (Si) produced
FIG. 1b shows the UV-vis spectrum of the inventive product sample 4 (P4), silicon particles of size 10-50 nm with median about 35 nm, and the inventive product 3 (P3) silicon particles of size 10-50 nm with median about 25 nm.
FIG. 2: Refractive index of silicon as a function of wavelength (λ), spectrum e: refractive index of silicon, line f: refractive index n=4.
FIG. 3a shows the silicon-containing particles according to Working Example 2a.
FIG. 3b shows silicon-containing particles according to Working Example 2b.
FIG. 1b shows the UV-vis absorption characteristics of a reference sample and of silicon of different primary particle size and different cluster size. In the direction of the arrow (increasing particle size), the absorption increases with increasing particle size within the wavelength range from about 400 nm to well over 1000 nm. Assignment of the spectra in FIG. 1b: spectrum d: 20131007-P6/2 mm, spectrum b: TiO₂-P25, spectrum a: 20130912-P4: sample 4, spectrum c: 20130912-P3: sample 3.

All figures based on a volume are in the unit of measurement of standard litres. The plasma reactor was operated at 0.45 kW and about 14 kHz with high-voltage pulses having a half-height width, abbreviated to the unit “t(50)” known to those skilled in the art, of 567 ns and a mass flow rate of 43 1 (STP)/min. For the plasma processes which follow, gas discharges conducted in a non-thermal equilibrium were used.

The free-space reactor used in the examples, abbreviated to “FSR”, had a tangential wall flow. The FSR was equipped with an arrangement for temperature measurement in the reactor. The geometry of a preferred reactor is specified hereinafter. The reactor tube had an external diameter of 36.1 mm and an internal diameter of 33.1 mm. The heating zone was provided for a length of 700 mm at a temperature of 1300° C.

The shielding tube for the temperature measurement probe had an external diameter of 6.7 mm, an internal diameter of 3.7 mm, and a temperature sensor for a measurement of high temperatures up to 2000° C. For a circular gas flow in the FSR, a tangential feed was provided. Table 1 discloses measurement points for the temperature value in the tubular reactor.

TABLE 1
Temperature curve as a function of measurement
point (x value in mm) in the reactor

| Measurement point | X value (mm) | Temperature value |
|---|---|---|
| M1 | −50 | RT |
| M2 | 0 | 595 |
| M3 | 50 | 926 |
| M4 | 100 | 1078 |
| M5 | 150 | 1149 |
| M6 | 200 | 1199 |
| M7 | 250 | 1227 |
| M8 | 300 | 1239 |
| M9 | 350 | 1238 |
| M10 | 400 | 1222 |
| M11 | 450 | 1186 |
| M12 | 500 | 1120 |
| M13 | 550 | 929 |
| M14 | 600 | 553 |
| M15 | 650 | RT |

### Working Example 1

Obtaining amorphous silicon particles in high-purity form in a free-space reactor. Monosilane was decomposed in an H₂ matrix (60% by volume). The hydrogen was used as heat transferer and as diluent gas. The residence time in the reactor tube, which had a length of 50 cm, was 100 to 400 milliseconds.

The cooling and removal of the amorphous silicon is effected in a fluid. In the present case, the decomposition products were passed through liquid paraffin. It was possible to stabilize the amorphous silicon primary particles formed in the paraffin and, according to the production conditions and concentration, they were in the form of clusters of aggregated primary particles.
The process conditions were:
(a) Gas mixture 2 standard litres (1 (STP)/min) of argon, 1 1 (STP)/min of argon with 5% by weight of SiH₄. The residence time was 100 ms. The sample 3 was obtained.
(b) 2 1 (STP)/min of argon and 2 1 (STP)/min of argon with 5% by weight of SiH₄. The residence time was 400 ms. The sample 4 was obtained.
This resulted in different primary particle sizes.
Under the process conditions (a), a primary particle size, for which the abbreviation d₅₀ known to those skilled in the art is used, of 20 to 25 nm was obtained. Under the conditions (b), the result was a primary particle size (d₅₀) of 35 to 40 nm.

The absorption of the amorphous silicon particles having a particle size of 20 to 25 nm, in the range from 400 to 1050 nm, in an emulsion in ethanol was less than 1. The absorption fell to below 0.5% at wavelengths above 500 nm and reached a value of about 0.1% at about 850 nm. The particle sizes of (a) and (b) had an absorption of greater than or equal to 1 at wavelengths below 400 nm. The silicon primary particles obtained were essentially amorphous and were transparent according to the primary particle size.

The absorption of the particles having a particle size of 35 to 40 nm was below 1 from 400 nm upwards and decreased to 0.7 at a wavelength of 1050 nm.

The absorption as a function of wavelength for the particles obtained in accordance with the invention is shown in FIG. 1b as the line form of the sample “TiO₂-P25”. Compared to known TiO₂ particles, the nanoparticulate silicon according to the invention, having particle sizes of 20 to 25 nm, shows much more favourable transmission properties than TiO₂ particles in the wavelength range from 400 to 740 nm and especially to 1100 nm.

It was also found that, as a result of doping of the nanoparticulate silicon with selenium, the particles according to the invention are effective as free-radical scavengers.

### Working Examples 2a and 2b

Plasma

The process conditions were as in Working Example 1, but with the following differences.
(2a) Monosilane was used together with about 10 ppm of selenium. The non-thermal plasma had a matrix composed of argon and 1% xenon.

Xenon promotes the homogeneity of discharge in the reactor space.

The median primary particle size of the silicon-comprising particles obtained in accordance with the invention was 5 to 10 nm. FIG. 3a shows a TEM image of these particles.
(2b) like (2a), but with the addition of 1% by volume of oxygen and extended residence times.

The median primary particle size of the silicon-comprising particles obtained in accordance with the invention, which had an SiO₂ shell, was about 40 nm. FIG. 3b shows a TEM image of these particles.

European patent application EP14195302 filed Nov. 28, 2014, is incorporated herein by reference.

Numerous modifications and variations on the present invention are possible in light of the above teachings. It is therefore to be understood that within the scope of the appended claims, the invention may be practiced otherwise than as specifically described herein.

1. A method for protection of a cell from electromagnetic radiation, the method comprising:
contacting the cell with a silicon-containing particle to absorb said electromagnetic radiation;
wherein said electromagnetic radiation has a wavelength range of from 10 to 1500 nm.

2. The method according to claim 1, wherein the particles
a) absorb electromagnetic radiation in the wavelength range of from greater than or equal to 10 nm to 1100 nm, and/or
b) absorb electromagnetic radiation in the UV range.

3. The method according to claim 1, wherein the silicon-containing particles have a content of greater than or equal to 50% to 100% by weight of silicon and a primary particle size of 1 to 500 nm.

4. The method according to claim 1, wherein the silicon-containing particles are amorphous particles.

5. The method according to claim 1, wherein the silicon-containing particles are amorphous particles of silicon.

6. The method according to claim 1, wherein the silicon-containing particles have a crystallinity of less than or equal to 2%.

7. The method according to claim 1, wherein the silicon-containing particles have
a) primary particles of size from 1 nm to 200 nm, and/or
b) a median primary particle size d₅₀=15 to 40 nm.

8. The method according to claim 1, wherein the particles comprise 60% to 100% by weight of silicon and, if the silicon content is less than 100% by weight, the remainder based on 100% by weight of the particles comprises carbon and optionally oxygen, and
wherein the particles are optionally transparent.

9. The method according to claim 1, wherein the particles have the following characteristics of electromagnetic radiation absorption:
a) with a primary particle size of 10 to 50 nm, the absorption in the wavelength range from 250 to 400 nm as compared with the absorption in the wavelength range from 400 to 750 nm is equal with a deviation of +/−40%, and/or
b) with a primary particle size of 10 to 50 nm, the ratio of the absorption in the wavelength range from (i) 250 to 400 nm as compared with the absorption at a wavelength of (ii) 550 nm is about (i):(ii)=2:1 to 8:1 with a deviation of +/−40%, and/or
c) with a primary particle size of 10 to 50 nm, the ratio of the absorption in the wavelength range from (i) 250 to 400 nm as compared with the absorption at a wavelength of (ii) 500 nm is about (i):(ii)=1.5:1 to 8:1 with a deviation of +/−30%, and/or
d) with a primary particle size of 10 to 50 nm, the ratio of the absorption in the wavelength range from (i) 250 to 350 nm as compared with the absorption at a wavelength of (ii) 450 nm is about (i):(ii)=2:1 to 4:1 with a deviation of +/−30%, and/or
e) with a primary particle size of 10 to 50 nm, the ratio of the absorption at a wavelength of (i) 250 nm as compared with the absorption at a wavelength of (ii) 450 nm is about (i):(ii)=2:1 to 4:1 with a deviation of +/−30%, and/or
f) with a primary particle size of 10 to 50 nm, the ratio of the absorption at a wavelength of (i) 350 nm as compared with the absorption at a wavelength of (ii) 450 nm is about (i):(ii)=2:1 to 3:1 with a deviation of +/−30%.

10. The method according to claim 1, wherein the silicon-containing particles have a refractive index (n) of greater than or equal to 3.0 at a wavelength of 500 to 2500 nm and/or a refractive index (n) of greater than or equal to 4.0 at a wavelength of 200 to 500 nm.

11. A method for increasing the refractive index of a cosmetic, dental, medical or pharmaceutical formulation, said method comprising:
contacting the cosmetic, dental, medical or pharmaceutical formulation with a silicon-containing particle which absorbs electromagnetic radiation which has a wavelength range of from 10 to 1500 nm.

12. A biocompatible UV protection composition and/or biodegradable UV protection composition, comprising:
silicon-containing particles, comprising primary particles of 5 to 100 nm and optionally clusters of these primary particles, having a silicon content of greater than or equal to 90% by weight to 100% by weight;
wherein said particles absorb electromagnetic radiation in the UV range.

13. A process for producing silicon-containing particles, said process comprising:
a) decomposing at least one gaseous silicon compound or one which is gaseous at elevated temperature,
b) optionally in the presence of at least one gas which is reactive under the reaction conditions or of a mixture of reactive gases,
c) in the presence of a diluent gas in an essentially oxygen-free atmosphere under (i) thermal conditions and/or (ii) in a plasma, and
d) effecting deposition with a fluid as silicon-containing particles.

14. The process according to claim 13, wherein the silicon-containing particles are deposited with a fluid in the form of a solution, emulsion, suspension, gel, foam, aerosol, smoke, or in colloidal form in a solution.

15. The process according to claim 13, wherein the gaseous silicon compound comprises hydrogen-containing silanes such as monosilane, disilane, trisilane and mixtures comprising at least one of the silanes.

16. A formulation, comprising:
amorphous silicon-containing particles having a content of greater than or equal to 50% to 100% by weight of silicon and a content of carbon and optionally oxygen and a median primary particle size of 5 to 500 nm, wherein the particles are in the form of clusters of primary particles, or
silicon-containing particles obtained by a process according to claim 13, wherein said formulation is a cosmetic, medical or pharmaceutical formulation for UV protection of the skin or hair.

17. The formulation according to claim 16, which comprises at least one auxiliary.
